(19) United States
(12) Patent Application Publication
(10) Pub. No.: US 2023/0127704 A1
(43) Pub. Date: Apr. 27, 2023

(54) CROSS-LINKED COMPOSITIONS AND THEIR USE

(71) Applicant: VERTELLUS HOLDINGS LLC, Indianapolis, IN (US)

(72) Inventors: William J. DOWNEY, Somerset, NJ (US); John H. YAMAMOTO, Kingston, Ontario (CA); Melissa H. JAIME, Blairstown, NJ (US); William A. KERNICK, III, Flanders, NJ (US)

(21) Appl. No.: 17/918,027

(22) PCT Filed: Apr. 9, 2021
(86) PCT No.: PCT/US2021/026582

§ 371 Date: Oct. 10, 2022

Related U.S. Application Data
(60) Provisional application No. 63/007,490, filed on Apr. 9, 2020.

(51) Int. Cl. C08L 71/02; A61K 8/73; A61K 8/26; A61K 8/19; A61Q 19/00

(52) U.S. Cl. CPC C08L 71/02; A61K 8/73; A61K 8/26; A61K 8/19; A61Q 19/00; A61K 2800/10; A61K 2800/412

(57) ABSTRACT

The present disclosure relates to chemical processes and more particular to chemical processes for forming cross-linked compositions and their use.

### CROSS-REFERENCE TO RELATED APPLICATIONS

This application claims the benefit of U.S. Provisional Patent Application No. 63/007,490, filed Apr. 9, 2020, the entire disclosure of which is hereby incorporated by reference.

### TECHNICAL FIELD

The present disclosure relates to chemical processes and more particularly to chemical processes for forming cross-linked compositions and their use.

### BACKGROUND

A variety of compounds and materials are used in formulating in the food, personal care, and pharmaceutical industries. A wide range of stabilizers, excipients, demulcents, binders, texturizers, fillers, colorants, and the like are known and applied to formulating food products, personal care products, and pharmaceutical products. An example of such materials is inorganic compounds or elements which find a wide range of uses in the food, personal care, and pharmaceutical industries. It is often the case that a formulation uses several different active and inactive ingredients having widely varying physical and chemical properties. For example, in some applications, an active ingredient is formulated with one or more inactive ingredients, such as stabilizers, excipients, demulcents, binders, texturizers, fillers, binders, colorants, and the like. In some applications, the physical properties of the various ingredients in a formulation can vary widely. For example, some pharmaceutical or personal care formulations include both water-soluble and water-insoluble ingredients. In some formulations, one or more active ingredients can be water soluble, while one or more inactive ingredients can be either water soluble (e.g., acacia gum) or water insoluble (e.g., colorants).

Restrictions on the use ingredients can require companies to spend large amounts of time and money to reformulate products. For example, recent restrictions on the use of borax in personal care items, has required manufacturers to redesign product formulations. Such product redesign can be challenging when a product's physical appearance and feel is known to and accepted by the public, or when the product has been approved by a regulatory authority under a specific formulation.

One of the challenges of formulation and reformulation chemistry can be the ability to process efficiently materials. For example, some ingredients used in food, personal care, or pharmaceutical formulations have poor flowability in processing. In such cases, the manufacture of a final product can become challenging and more expensive.

As such, there is a need for improved formulation compositions and methods for their preparation.

### SUMMARY

In one aspect, the disclosure provides a composition comprising a. a stabilizer, demulcent, binder, emulsifier, or texturizer; b. a water-insoluble inorganic; and c. a cross-linking agent.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A composition comprising

a. a colloidal material;

b. a water-insoluble inorganic; and

c. a cross-linking agent.

2. The composition of clause 1, wherein the colloidal material is a stabilizer, a demulcent, a binder, an emulsifier, or a texturizer.

3. The composition of clause 1 or 2, wherein the colloidal material is selected from the group consisting of gelatin, casein, albumin, agar, dextrin, sugar, welan gum, xanthan gum, guar gum, acacia gum, corn fiber gum, peach gum, locust bean gum, gum rosin, tamarind gum, tragacanth gum, flax seed gum, carrageenan gum, gum arabic, tara gum, and sesbania gum.

4. The composition of any one of clauses 1 to 3, wherein the water-insoluble inorganic is selected from the group consisting of dibasic calcium phosphate, calcium phosphate tribasic, dicalcium sulfate, iron oxide, iron oxide yellow, magnesium oxide, magnesium carbonate, barium sulfate, silicon, sulfur, and zinc oxide.

5. The composition of any one of the preceding clauses, wherein the cross-linking agent is selected from the group consisting of sodium aluminate, calcium aluminate, magnesium aluminate, barium aluminate and strontium aluminate.

6. The composition of any one of the preceding clauses, wherein the composition has a basic flow energy of less than about 150 mJ.

7. The composition of any one of the preceding clauses, wherein the composition has a basic flow energy of less than about 125 mJ.

8. The composition of any one of the preceding clauses, wherein the composition has a basic flow energy of less than about 100 mJ.

9. The composition of any one of the preceding clauses, wherein the composition has a basic flow energy of less than about 85% of a basic flow energy of the water-insoluble inorganic in the absence of the colloidal material and the cross-linking agent.

10. The composition of any one of the preceding clauses, wherein the composition has a basic flow energy of less than about 75% of a basic flow energy of the water-insoluble inorganic in the absence of the colloidal material and the cross-linking agent.

11. The composition of any one of the preceding clauses, wherein the composition has a conditioned bulk density of less than about 97% of a conditioned bulk density of the water-insoluble inorganic in the absence of the colloidal material and the cross-linking agent.

12. The composition of any one of the preceding clauses, wherein the composition has a conditioned bulk density of less than about 95% of a conditioned bulk density of the water-insoluble inorganic in the absence of the colloidal material and the cross-linking agent

13. The composition of any one of the preceding clauses, wherein the composition has a volume moment mean particle size of from about 10 to about 50 microns.

14. The composition of any one of the preceding clauses, wherein the composition has a volume moment mean particle size of from about 25 to about 50 microns.

15. The composition of any one of the preceding clauses, wherein the water-insoluble inorganic is in an amount of from about 65 wt % to about 85 wt %.

16. The composition of any one of the preceding clauses, wherein the water-insoluble inorganic is in an amount of from about 70 wt % to about 80 wt %.

17. The composition of any one of the preceding clauses, wherein the colloidal material is present in an amount of from about 13 wt % to about 35 wt %.

18. The composition of any one the preceding clauses, wherein the colloidal material is present in an amount of from about 20 wt % to about 25 wt %.

19. The composition of any one of the preceding clauses, wherein the cross-linking agent is in an amount of from about 0.3 wt % to about 1.5 wt %.

20. The composition of any one of clauses 1 to 18, wherein the cross-linking agent is in an amount of from about 2 wt % to about 4 wt % relative to the colloidal material.

21. The composition of any one of the preceding clauses, further comprising water in an amount of from about 1 wt % to about 3 wt %.

22. The composition of clause 21, wherein the water-insoluble inorganic is in an amount of from about 63 wt % to about 85 wt %.

23. The composition of clause 21 or 22, wherein the cross-linking agent is in an amount of from about 0.3 wt % to about 1.5 wt %.

24. The composition of any one of clauses 21 to 23, wherein the cross-linking agent is in an amount of from about 2 wt % to about 4 wt % relative to colloidal material.

25. The composition of any one of clauses 21 to 24, wherein the colloidal material is present in an amount of from about 13 wt % to about 34 wt %.

26. An aqueous composition comprising

a. a colloidal material;

b. a water-insoluble inorganic;

c. a cross-linking agent; and

d. water.

27. The aqueous composition of clause 26, wherein the water is in an amount of from about 40 wt % to about 70 wt %.

28. The aqueous composition of clause 26 or 27, wherein colloidal material is a stabilizer, a demulcent, a binder, an emulsifier, or a texturizer.

29. The aqueous composition of any one of clauses 26 to 28, wherein the colloidal material is selected from the groups consisting of gelatin, casein, albumin, agar, dextrin, sugar, welan gum, xanthan gum, guar gum, acacia gum, corn fiber gum, peach gum, locust bean gum, gum rosin, tamarind gum, tragacanth gum, flax seed gum, carrageenan gum, gum arabic, tara gum, and sesbania gum.

30. The aqueous composition of any one of clauses 26 to 29, wherein the water-insoluble inorganic is selected from the group consisting of dibasic calcium phosphate, calcium phosphate tribasic, dicalcium sulfate, iron oxide, iron oxide yellow, magnesium oxide, magnesium carbonate, barium sulfate, silicon, sulfur, and zinc oxide.

31. The aqueous composition of any one of clauses 26 to 30, wherein the cross-linking agent is selected from the group consisting of sodium aluminate, calcium aluminate, magnesium aluminate, barium aluminate and strontium aluminate.

32. The aqueous composition of any one of clauses 26 to 31, wherein the water-insoluble inorganic is in an amount of from about 19 wt % to about 51 wt %.

33. The aqueous composition of any one of clauses 26 to 32, wherein the water-insoluble inorganic is in an amount of from about 19 wt % to about 40 wt %.

34. The aqueous composition of any one of clauses 26 to 33, wherein the colloidal material is present in an amount of from about 4 wt % to about 21 wt %.

35. The aqueous composition of any one of clauses 26 to 34, wherein the cross-linking agent is in an amount of from about 0.1 wt % to about 1 wt %.

36. The aqueous composition of any one of clauses 26 to 35, wherein the cross-linking agent is in an amount of from about 2.0 wt % to about 4.0 wt % relative to the colloidal material.

37. A personal care composition comprising the composition of any one of clauses 1 to 25 or the aqueous composition of any one of clauses 26 to 36, and a base composition.

38. The personal care composition of clause 37, wherein the base composition is a lotion, a cream, a face mask, a conditioner, or a serum.

39. The personal care composition of clause 37 or 38, wherein the base composition is in the form of an emulsion, a surfactant system, or an aqueous gel.

40. The personal care composition of clause 37 or 38, wherein the personal care composition comprises a base composition selected from the group consisting of a lotion, a cream, a face mask, a conditioner, and a serum, and the base composition is in the form of an emulsion.

41. The personal care composition of any one of clauses 37 to 39, wherein the personal care composition comprises a base composition selected from the group consisting of a body wash, a facial cleanser, or a shampoo, and the base composition is in the form of a surfactant system.

42. The personal care composition of any one of clauses 37 to 39, wherein the personal care composition comprises a base composition that is a serum, and the base composition is an aqueous gel.

### BRIEF DESCRIPTION OF THE DRAWINGS

FIG. 1 is a chart showing the viscosity effect of several tested crosslinkers on an acacia gum solution.

FIG. 2 is a chart showing the viscosity effect of sodium aluminate and borax at various concentrations on a 25% acacia gum solution.

FIGS. 3A-C are a set of images of 10 mil draw-downs of 4% iron oxide colloid composition compared to a non-colloidal composition. FIG. 3A shows a 4% iron oxide colloid (left) compared to a non-colloidal 4% iron oxide composition; FIG. 3B shows a microscope image of the 4% iron oxide colloid; FIG. 3C shows a microscope image of the non-colloidal 4% iron oxide composition; FIG. 3D shows the particle size distribution of the iron oxide colloid; and FIG. 3E shows the particle size distribution of the iron oxide particles.

FIGS. 4A-C are a set of images of 10 mil draw-downs of a 5.33% sulfur colloid composition compared to a non-colloidal 4% sulfur composition. FIG. 4A shows a 5.33% sulfur colloid (left) compared to a non-colloidal 4% sulfur composition; FIG. 4B shows a microscope image of the 5.33% sulfur colloid; and FIG. 4C shows the non-colloidal 4% sulfur composition.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemicals embraced by the present disclosure and are disclosed herein just as if each and every combination was individually and explicitly disclosed. In addition, all subcombinations of the chemicals listed in the embodiments describing such variables are also specifically embraced by the present disclosure and are disclosed herein just as if each and every such sub-combination of chemicals was individually and explicitly disclosed herein.

In some embodiments, the compositions described herein comprise a. a colloidal material; b. a water-insoluble inorganic; and c. a cross-linking agent.

In some embodiments, the compositions described herein comprise a. a stabilizer, demulcent, binder, emulsifier, or texturizer; b. a water-insoluble inorganic; and c. a cross-linking agent.

In some embodiments, the composition of the disclosure can be a colloid. In some embodiments, a colloid as described herein comprises a. a colloidal material; b. a water-insoluble inorganic; and c. a cross-linking agent. In some embodiments, a colloid as described herein comprises a. a stabilizer, demulcent, binder, emulsifier, or texturizer; b. a water-insoluble inorganic; and c. a cross-linking agent. In some embodiments, a colloid as described herein comprises a. a colloidal material that can be a stabilizer, a demulcent, a binder, an emulsifier, a texturizer, or a combination thereof; b. a water-insoluble inorganic; and c. a cross-linking agent.

It will be appreciated that the colloidal material used in the compositions described herein is not particularly restricted, and can be colloidal material known in the food, personal care, or pharmaceutical industry. It will be appreciated that the stabilizer, demulcent, binder, emulsifier, or texturizer used in the compositions described herein is not particularly restricted, and can be any stabilizer, demulcent, binder, emulsifier, or texturizer known in the food, personal care, or pharmaceutical industry. In some embodiments, the stabilizer, demulcent, binder, emulsifier, or texturizer can be a colloidal material. As used herein, “colloidal material” refers to material that functions to suspend particulate material in a solution. It will be understood that colloidal materials are polysaccharides composed of sugar molecules that contain carboxy and hydroxyl groups. These colloidal materials will have a molecular weight greater than 1×10⁵ Da, especially between 2×10⁵ to 5×10⁶ Da. Exemplary colloidal materials include, but are not limited to bio-gums, edible gums, natural gums, vegetable gums, starch gums, and polysaccharide gums. Examples of colloidal materials include but are not limited to gelatin, casein, albumin, agar, dextrin, sugar, welan gum, xanthan gum, guar gum, acacia gum, corn fiber gum, peach gum, locust bean gum, gum rosin, tamarind gum, tragacanth gum, flax seed gum, carrageenan gum, gum arabic, tara gum, and sesbania gum. In some embodiments, the stabilizer, demulcent, binder, emulsifier, or texturizer can be a colloidal material that is acacia gum.

In some embodiments, the colloidal material is in an amount of from about 13 wt % to about 35 wt % on a solids basis of the compositions described herein. In some embodiments, the colloidal material is in an amount of from about 15 wt % to about 30 wt % on a solids basis of the compositions described herein. In some embodiments, the colloidal material is in an amount of from about 20 wt % to about 25 wt % on a solids basis of the compositions described herein. In some embodiments, the colloidal material is in an amount of any range within the range of about 13 wt % to about 35 wt % on a solids basis of the compositions described herein.

In some embodiments, the stabilizer, demulcent, binder, emulsifier, or texturizer is in an amount of from about 13 wt % to about 35 wt % on a solids basis of the compositions described herein. In some embodiments, the stabilizer, demulcent, binder, emulsifier, or texturizer is in an amount of from about 15 wt % to about 30 wt % on a solids basis of the compositions described herein. In some embodiments, the stabilizer, demulcent, binder, emulsifier, or texturizer is in an amount of from about 20 wt % to about 25 wt % on a solids basis of the compositions described herein. In some embodiments, the stabilizer, demulcent, binder, emulsifier, or texturizer is in an amount of any range within the range of about 13 wt % to about 35 wt % on a solids basis of the compositions described herein.

It will be appreciated that the water-insoluble inorganic used in the compositions described herein is not particularly restricted, and can be any water-insoluble inorganic commonly used in the food, personal care, or pharmaceutical industries. Examples of suitable water-insoluble inorganics for use in the embodiments described herein include, but are not limited to, calcium inorganics, such as dibasic calcium phosphate (an excipient used as a binder or filler), calcium phosphate tribasic (an excipient used as a binder or filler), dicalcium sulfate, and the like, fluoride, iodine, iron inorganics, such as iron oxide (an excipient or a colorant), iron oxide yellow (an excipient or a colorant), and the like, magnesium inorganics, such as magnesium oxide (a diluent), magnesium carbonate (a diluent), and the like, manganese inorganics, molybdenum inorganics, phosphorus, silicon, sulfur, and zinc inorganics, such as zinc oxide (an excipient used as a color additive), barium sulfate (an excipient). In some embodiments, the water-insoluble inorganic is in an amount of from about 65 wt % to about 85 wt % on a solids basis of the compositions described herein. In some embodiments, the water-insoluble inorganic is in an amount of from about 70 wt % to about 80 wt % on a solids basis of the compositions described herein. In some embodiments, the water-insoluble inorganic is in an amount of from about 75 wt % to about 80 wt % on a solids basis of the compositions described herein. In some embodiments, the water-insoluble inorganic is in an amount of any range within the range of about 65 wt % to about 85 wt % on a solids basis of the compositions described herein.

The water-insoluble inorganics may be in the form of a powder. These water-insoluble inorganic powders are available in a variety of particle sizes and particle size distributions. Particle size as used herein can reference median particle size by volume, or volume moment mean D[4,3], as specified. An exemplary reference for particle size definition and how it will be measured is the Malvern Instruments white paper entitled “A basic guide to particle characterization.” Particle size is reported as measured by a Malvern Instruments Particle Analyzer or similar instrumentation. The compositions described herein are applicable to water-insoluble inorganics with median particle size by volume ranging from 5 to 100 microns in diameter. Some materials, such as magnesium oxide, are readily available in narrow particle size distributions having median particle sizes of 10 or 50 microns by volume. Others, such as sulfur, typically have much broader particle size distributions ranging from 2 to 110 microns with a median particle size by volume of 25 to 45 microns.

It will be appreciated that the cross-linking agent used in the compositions described herein is not particularly restricted, and can be any cross-linking agent that functions as a clumping or crosslinking material by linking the hydroxyl and carboxy groups of a stabilizer, demulcent, binder, emulsifier, or texturizer, such as a colloidal material. Exemplary cross-linking agents include, but are not limited to, borates, chromates, titanates, zirconates and aluminates. In some embodiments, cross-linking agents can be metal aluminates based on alkali metals or alkaline earth metals. Examples of cross-linking agents include, but are not limited to, sodium aluminate, calcium aluminate, magnesium aluminate, barium aluminate and strontium aluminate. In some embodiments, the cross-linking agent is calcium aluminate. In some embodiments, the cross-linking agent is magnesium aluminate. In some embodiments, the cross-linking agent is barium aluminate. In some embodiments, the cross-linking agent is strontium aluminate. In some embodiments, the cross-linking agent is sodium aluminate. In some embodiments, the cross-linking agent is in an amount of from about 0.3 wt % to about 1.5 wt % on a solids basis of the compositions described herein. In some embodiments, the cross-linking agent is in an amount of from about 0.5 wt % to about 1 wt % on a solids basis of the compositions described herein. In some embodiments, the cross-linking agent is in an amount of from about 0.7 wt % to about 1 wt % on a solids basis of the compositions described herein. In some embodiments, the cross-linking agent is in an amount of any range within the range of about 0.3 wt % to about 1.5 wt % on a solids basis of the compositions described herein.

In some embodiments, the cross-linking agent is in an amount of from about 2.0 wt % to about 4.0 wt % relative to the stabilizer, demulcent, binder, emulsifier, or texturizer. In some embodiments, the cross-linking agent is in an amount of from about 2.5 wt % to about 3.5 wt % relative to the stabilizer, demulcent, binder, emulsifier, or texturizer. In some embodiments, the cross-linking agent is in an amount of from about 2.0 wt % to about 3.0 wt % relative to the stabilizer, demulcent, binder, emulsifier, or texturizer. In some embodiments, the cross-linking agent is in an amount of any range within the range of about 2.0 wt % to about 4.0 wt % relative to the stabilizer, demulcent, binder, emulsifier, or texturizer.

In some embodiments, the cross-linking agent is in an amount of from about 2.0 wt % to about 4.0 wt % relative to the colloidal material. In some embodiments, the cross-linking agent is in an amount of from about 2.5 wt % to about 3.5 wt % relative to the colloidal material. In some embodiments, the cross-linking agent is in an amount of from about 2.0 wt % to about 3.0 wt % relative to the colloidal material. In some embodiments, the cross-linking agent is in an amount of any range within the range of about 2.0 wt % to about 4.0 wt % relative to the colloidal material.

In some embodiments, the compositions described herein comprise an amount of water. In some embodiments, the water is in an amount of up to about 3 wt % of a solid composition. In some embodiments, the water is in an amount of from about 1 wt % to about 3 wt % of a solid composition. It will be appreciated that the amounts of the various components in the compositions described herein can be in a weight percent amount measured on a solids basis in the composition, or in an amount of the absolute weight percent of the various components in the composition. In some embodiments, where a composition, such as a spray-dried composition, is anhydrous, it will be appreciated that the solids basis of a composition can be about equivalent to the absolute. In some embodiments, where a composition, such as a spray-dried composition, contains water in an amount of up to about 3 wt % of the composition, the values of the amounts of the various components in the compositions described herein can be in a weight percent amount measured on a solids basis in the composition factoring in the amount of water. In some embodiments, where a composition, such as a spray-dried composition, contains water in an amount of up to about 3 wt % of the composition, the values of the amounts of the various components in the compositions described herein can be in a weight percent amount measured on an absolute weight basis of the composition including the amount of water in the composition.

In some embodiments, the compositions described herein have a basic flow energy. Illustratively, basic flow energy is a measurement of a composition's resistance to flow. For example, a composition with a higher basic flow energy may resist flow more in an auger system than a composition with a lower basic flow energy. As such, improvements in basic flow energy may provide improved metering and processing for a powder. In some embodiments, a composition described herein has a basic flow energy of less than about 150 mJ, less than about 125 mJ, or less than about 100 mJ, as measured by a Freeman Technology FT4 powder rheometer. In some embodiments, the basic flow energy of a composition comprising a colloidal material, a cross-linker and a water-soluble inorganic is less than the basic flow energy of a composition comprising the water-insoluble inorganic without the colloidal material and the cross-linking agent. In some embodiments, the composition has a basic flow energy of less than about 85% or less than about 70% of a basic flow energy of the water-insoluble inorganic in the absence of the colloidal material and the cross-linking agent.

In some embodiments, the compositions described herein have a conditioned bulk density. Measured at a low-stress, homogeneous packing state, conditioned bulk density is representative of the normal condition of a powder without the influence of localized stress and excess air. A decrease in conditioned bulk density can be indicative of particle deagglomeration, producing a more uniform particle size distribution as compared to the bi-modal distribution typically observed in powder samples with a high tendency to agglomerate. As such, improvements in conditioned bulk density may provide improved dispersability performance. In some embodiments, a composition described herein has a conditioned bulk density of less than about 0.8 g/mL, less than about 0.75 g/mL, or less than about 0.7 g/mL, as measured by Freeman Technology FT4 powder rheometer. In some embodiments, the conditioned bulk density of a composition comprising a colloidal material, a cross-linker and a water-soluble inorganic is less than the conditioned bulk density of a composition comprising the water-insoluble inorganic without the colloidal material and the cross-linking agent. In some embodiments, the composition has a conditioned bulk density of less than about 97% or less than about 95% of a conditioned bulk density of the water-insoluble inorganic in the absence of the colloidal material and the cross-linking agent.

In some embodiments, compositions of the disclosure, such as colloid compositions described herein, can be prepared as aqueous slurries. In some embodiments, the aqueous composition comprises from about 40% water to about 70% water by weight. In some embodiments, the aqueous composition comprises from about 40% water to about 60% water by weight. In some embodiments, the aqueous composition comprises about 50% water by weight. In some embodiments, the aqueous composition comprises about 70% water by weight. It will be appreciated that the amounts of the various components in the compositions described herein can be in a weight percent amount measured on a solids basis in the composition, or in an amount of the absolute weight percent of the various components in the composition as described herein.

It will be appreciated that compositions of the disclosure, such as colloid compositions described herein, can be useful in various applications that require a cross-linked composition of a water-insoluble inorganic. In some embodiments, compositions of the disclosure, such as colloid compositions described herein, may be used in a personal care item (a.k.a. a personal care composition), such as those known in the art as skin care products, anti-perspirants, deodorants, cosmetics, hair care products, and the like. In some embodiments, the compositions of the disclosure, such as colloid compositions described herein, can be in an amount of about 0.1 wt % to about 20 wt % of a personal care composition as described herein. It will be appreciated that the range of the amount of compositions of the disclosure, such as colloid compositions described herein, used in a personal care composition includes all possible ranges and values between 0.1 wt % and 20 wt %, such as 6 wt % to 20 wt %, 0.1 wt % to 15 wt %, 4 wt % to 10 wt %, 0.5 wt % to 10 wt %, 1 wt % to 10 wt %, 2 wt % to 14 wt %, 3 wt % to 13 wt %, 2 wt % to 5 wt %, and the like. The personal care item can have a primary use, such as for use as a moisturizer, a conditioner, an anti-aging composition, a skin lightener, a sunless tanner, a sunscreen, an anti-perspirant, a shave preparation, an after-shave, a foundation, a lipstick, a hair styling product, a shampoo, a cleanser, and the like. Personal care items can include a base composition, such as lotions, liquid soaps, creams, face masks, conditioners, serums, body washes, facial cleansers, shampoos, and the like. It will be appreciated that personal care items can be formulated in a variety of forms, such as a solution (water or oil based), a dispersion, an emulsion, an aqueous gel, a surfactant system, and the like. In some embodiments, the personal care item can be fluid or solid, such as gels, sticks, flowable solids, amorphous materials, and the like. In some embodiments, emulsion compositions comprise a water phase, one or more emulsifiers, one or more emollients, one or more humectants, one or more skincare actives (hyaluronic acid, niacinamide, allantoin, panthenol, peptides, etc), one or more rheology modifiers (synthetic and natural gums, etc), or one or more preservatives, other cosmetic raw materials, and the like. In some embodiments, surfactant systems comprise water, one or more anionic, amphoteric, non-ionic and cationic surfactant, one or more moisturizers, one or more humectants, one or more botanicals, one or more rheology modifiers, one or more preservatives, other cosmetic raw materials, and the like. In some embodiments, aqueous gels comprise water, one or more rheology modifiers, one or more moisturizers, one or more humectants (glycerin, glycols, skincare actives such as Hyaluronic acid, niacinamide, allantoin, panthenol, peptides, etc), one or more preservatives, other cosmetic raw materials, and the like. It will be appreciated that “other cosmetic raw materials” include those as outlined in in The Personal Care Product Council's International Cosmetic Ingredient Dictionary & Handbook, 16th Edition.

It will be further appreciated that applications of the compositions described herein can include the mixing of a composition of the disclosure, such as a colloid composition described herein, with a base composition in any of the forms described herein and a carrier to provide an end product. As used herein, a “carrier” means any carrier known in the art of personal care items, such as water (e.g. an aqueous carrier), water miscible solvents, oils, and the like.

Suitable carriers include water and/or water miscible solvents. The personal care item may comprise from about 1 wt % to about 95 wt % of water and/or water miscible solvent. The composition may comprise in wt % from about 1%, about 3%, about 5%, about 10%, about 15%, about 20%, about 25%, about 30%, about 35%, about 40%, about 45%, about 50%, about 55%, about 60%, about 65%, about 70%, about 75%, about 80%, or about 90% to about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5% water and/or water miscible solvents. Suitable water miscible solvents include monohydric alcohols, dihydric alcohols, polyhydric alcohols, glycerol, glycols, polyalkylene glycols such as polyethylene glycol, and mixtures thereof. Particularly suitable solvents include lower aliphatic alcohols such as ethanol, propanol, butanol, isopropanol, diols such as 1,2-propanediol, 1,3-propanediol, butanediol, pentanediol, hexanediol, heptanediol, decanediol; glycerin; water, and mixtures thereof. In certain embodiments, the personal care composition comprises water, diols, glycerin, and combinations thereof.

Suitable carriers also include oils. The personal care composition may comprise from about 0.1 wt % to about 95 wt % of one or more oils. The composition may comprise in wt % from about 0.1%, about 0.5%, about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, or 90% to about 95%, about 85%, about 80%, about 75%, about 70%, about 65%, about 60%, about 55%, about 50%, about 45%, about 40%, about 35%, about 30%, about 25%, about 20%, about 15%, about 10%, about 5%, or about 3% of one or more oils. Oils may be used to solubilize, disperse, or carry materials that are not suitable for water or water soluble solvents. Suitable oils include silicones, hydrocarbons, esters, amides, ethers, and mixtures thereof. Oils may be fluid at ambient conditions. However, certain personal care product forms (i.e., solid or semi-solid stick) may require non-fluid oils. The oils may be volatile or nonvolatile. “Nonvolatile” means a material that exhibits a vapor pressure of no more than about 0.2 mm Hg at 25° C. at one atmosphere and/or a material that has a boiling point at one atmosphere of at least about 300° C. “Volatile” means that the material exhibits a vapor pressure of at least about 0.2 mm Hg at 20° C. Volatile oils may be used to provide a lighter feel when a heavy, greasy film is undesirable.

Suitable oils include volatile oils. In certain embodiments, the volatile oils may have a viscosity ranging from about 0.5 to 5 centistokes at 25° C. Volatile oils may be used to promote more rapid drying of the skin care composition after it is applied to skin. Nonvolatile oils are also suitable for use in the composition. Nonvolatile oils are often used for emolliency and protective properties.

Other suitable oils include esters. The suitable esters typically contained at least 10 carbon atoms. These esters include esters with hydrocarbyl chains derived from fatty acids or alcohols (e.g., mono-esters, polyhydric alcohol esters, and di- and tri-carboxylic acid esters). The hydrocarbyl radicals of the esters hereof may include or have covalently bonded thereto other compatible functionalities, such as amides and alkoxy moieties (e.g., ethoxy or ether linkages). Exemplary esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oleyl myristate, lauryl acetate, cetyl propionate, C₁₂-C₁₅ alkyl benzoate, diisopropyl adipate, dibutyl adipate, and oleyl adipate. Other suitable esters are further described in The Personal Care Product Council's International Cosmetic Ingredient Dictionary & Handbook, 16th Edition., under the functional category of “Esters.” Other esters suitable for use in the personal care composition include those known as polyhydric alcohol esters and glycerides.

In some embodiments, the personal care item, including the base composition as described herein comprises various components known to one of skill in the art, such as humectants, thickeners, suspensing agents, emulsifiers, emollients, structuring agents, or pH adjusters.

An emulsifier can be particularly suitable when a base composition is in the form of an emulsion or if immiscible materials are being combined. The personal care item may comprise in wt % from about 0.05%, about 0.1%, about 0.2%, about 0.3%, about 0.5%, or about 1% to about 20%, about 10%, about 5%, about 3%, about 2%, or about 1% emulsifier. Emulsifiers may be nonionic, anionic or cationic. Non-limiting examples of emulsifiers are disclosed in U.S. Pat. Nos. 3,755,560, 4,421,769, and McCutcheon's, Emulsifiers and Detergents, 2010 Annual Ed., published by M. C. Publishing Co. Other suitable emulsifiers are further described in The Personal Care Product Council's International Cosmetic Ingredient Dictionary & Handbook, 16th Edition., under the functional category of “Surfactants—Emulsifying Agents.” Structuring agents can be used to increase viscosity, thicken, solidify, or provide solid or crystalline structure to a personal care item. Structuring agents are typically grouped based on solubility, dispersibility, or phase compatibility. Examples of aqueous or water structuring agents include polymeric agents, natural or synthetic gums, polysaccharides, and the like. In some embodiments, the composition comprises in wt % from about 0.0001%, about 0.001%, about 0.01%, about 0.05%, about 0.1%, about 0.5%, about 1%, about 2%, about 3%, about 5% to about 25%, about 20%, about 10%, about 7%, about 5%, about 4%, or about 2% of the composition, of one or more structuring agents.

Polysaccharides and gums may be suitable aqueous phase thickening agents. Suitable classes of polymeric structuring agents include but are not limited to carboxylic acid polymers, polyacrylamide polymers, sulfonated polymers, high molecular weight polyalkylglycols or polyglycerins, copolymers thereof, hydrophobically modified derivatives thereof, and mixtures thereof.

In some embodiments, the other components provided in a personal care item can include, but are not limited to, glycerin, xanthan gum, cetearyl alcohol/ceteareth-20 blend, glyceryl Stearate/PEG-100 stearate blend, dicapryl adipate, dimethicone. In some embodiments, the base composition comprises 10-15 wt % glycerin or other humectant such as propylene glycol, 2-5 wt % Xanthan Gum or other thickener/suspending agent such as acrylate copolymer or synthetic polymer, 48-62 wt % emulsifier such as PEG-150 disterate, Cetearteth-20, Ceteareth-25, glyceryl hydroxystearate, stearic acid, hydroxystearic acid, cetyl alcohol, stearyl alcohol, Emulsifying Wax NF, polysorbate 20, cetearyl alcohol, glyceryl stearate, PEG-100 stearate and/or blends of these, 20-30 wt % emollient such as dicapryl adipate, dimethicone, C₁₂-C₁₅ alkyl benzoate, caprylic capric triglycerides, isopropyl myristate, or isopropyl palmitate. In some embodiments, the wt % values provided above are of “dry” or “non-aqueous” values.

In some embodiments, the base composition comprises 1.0-5.0 wt % glycerin or other humectant such as propylene glycol, 0.1-0.75 wt % Xanthan Gum or other thickener/suspending agent such as acrylate copolymer or synthetic polymer, 3-10 wt % emulsifier such as PEG-150 disterate, Cetearteth-20, Ceteareth-25, glyceryl hydroxystearate, stearic acid, hydroxystearic acid, cetyl alcohol, stearyl alcohol, Emulsifying Wax NF, polysorbate 20, cetearyl alcohol, glyceryl stearate, PEG-100 stearate and/or blends of these, 1.0-5.0 wt % emollient such as dicapryl adipate, dimethicone, C₁₂-C₁₅ alkyl benzoate, caprylic capric triglycerides, isopropyl myristate, or isopropyl palmitate. In some embodiments, the personal care item can also contain from 60-80 wt % water.

In some embodiments, the base composition comprises 1.5-2.25 wt % glycerin or other humectant such as propylene glycol, 0.3-0.75 wt % Xanthan Gum or other thickener/suspending agent such as acrylate copolymer or synthetic polymer, 7.2-9.3 wt % emulsifier such as PEG-150 disterate, Cetearteth-20, Ceteareth-25, glyceryl hydroxystearate, stearic acid, hydroxystearic acid, cetyl alcohol, stearyl alcohol, Emulsifying Wax NF, polysorbate 20, cetearyl alcohol, glyceryl stearate, PEG-100 stearate and/or blends of these, 3.0-3.5 wt % emollient such as dicapryl adipate, dimethicone, C₁₂-C₁₅ alkyl benzoate, caprylic capric triglycerides, isopropyl myristate, or isopropyl palmitate. In some embodiments, the personal care item can also contain from 60-80 wt % water.

The compositions and products described herein can be demonstrated by way of the following non-limiting examples. It will be appreciated that a person skilled in the art will be able to modify the examples provided herein based on the descriptions provided with respect to the various embodiments to prepare alternative compositions and products.

### Example 1

Acacia Gum and cross-linker combinations were evaluated by dissolving 25 grams of the Gum in 75 grams of de-ionized water by mixing until the solution was clear, and then the crosslinker under evaluation was added slowly while heating to 76° C. Each solution was allowed to cool at room temperature for 24 hours. Solution viscosity was measured at 25° C. using a Brookfield LV DV-II+ Viscometer. A measurement of solution viscosity presented a significant difference between the prepared samples. As controls, a non-crosslinked solution was prepared and had a measured viscosity of 80 cP, while a comparative borax solution was prepared and had a measured viscosity of over 100,000 cP. Varying levels of each crosslinker ranging from 2% to 10% weight to weight of acacia gum in a 25% aqueous solution were evaluated to yield a similar increase in viscosity of the gum solution (FIG. 1). Only one additive produced a significant effect on the viscosity, which was sodium aluminate.

TABLE 1

| Coupling Agent | % weight to Gum | Viscosity (cP) |
|---|---|---|
| No crosslinker | 0.0 | 80 |
| borax | 2.0 | 137,000 |
| glutaraldehyde | 2.0 | 140 |
| glutaraldehyde | 10.0 | 3,000 |
| Calcium chloride | 4.0 | 80 |
| Calcium chloride | 10.0 | 40 |
| Citric acid | 10.0 | 50 |
| Sodium hexametaphosphate | 10.0 | 200 |
| Sodium trimetaphosphate | 10.0 | 300 |
| Zemac E-60 | 2.0 | 120 |
| Zemac E-60 | 10.0 | 85 |
| Zemac E-60 + H₂SO₄ | 10.0 | 350 |
| NaOH | 4.0 | 200 |
| NaOH | 10.0 | 40 |
| Glycerol + H₂SO₄ | 20.0 | 1040 |
| Sodium aluminate | 3.0 | 200,000 |

### Example 2

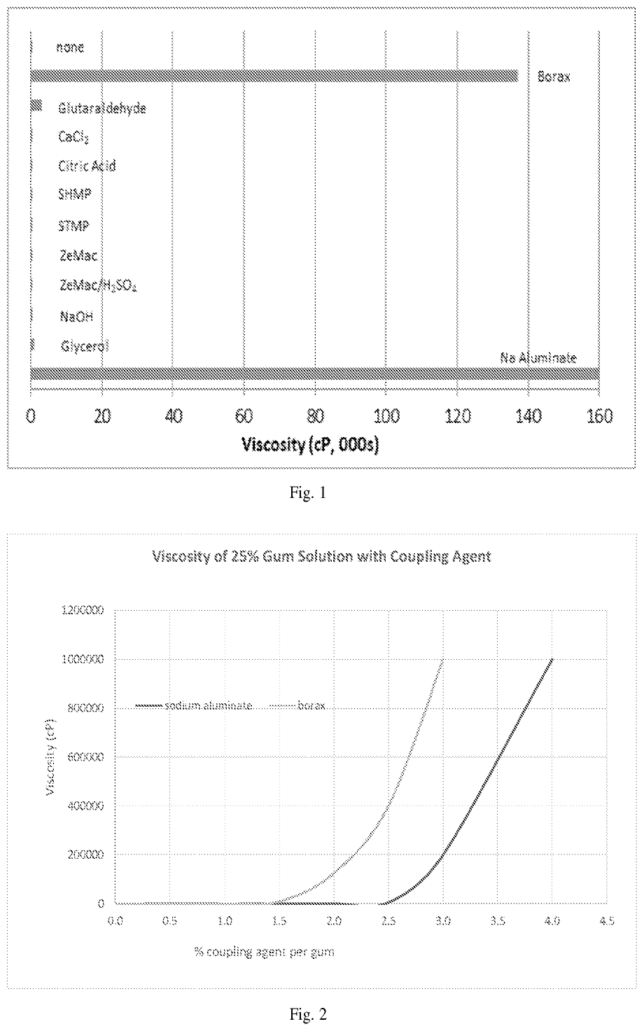

Using the method described in Example 1, a comparison of the viscosity build was made of various concentrations of sodium aluminate and borax added to a 25% acacia gum solution (Table 2 and FIG. 2). It was found that sodium aluminate can match the performance of borax by adjusting the loading level in the mixture.

TABLE 2

| | % Coupling Agent per Gum | | | | | | |
|---|---|---|---|---|---|---|---|
| | 0.0 | 1.0 | 1.5 | 2.0 | 2.5 | 3.0 | 4.0 |
| Ingredient | % Ingredient in Solution | | | | | | |
| Deionized | 75.00 | 74.75 | 74.625 | 74.50 | 74.375 | 74.25 | 74.00 |
| Water | | | | | | | |
| Acacia Gum | 25.00 | 25.00 | 25.00 | 25.00 | 25.00 | 25.00 | 25.00 |
| Coupling | 0.00 | 0.25 | 0.375 | 0.50 | 0.625 | 0.75 | 1.00 |
| Agent | | | | | | | |

### Example 3

The components shown in Table 3 were mixed with deionized water to prepare an aqueous slurry at 30 wt % solids. The slurry was spray dried using typical parameters for a water-based system to achieve a moisture content less than 3%. The resulting powder was analyzed for particle size distribution as measured by a Malvern Instruments Particle Analyzer, and moisture content. Conditioned Bulk Density (CBD) and Basic Flow Energy (BFE) were determined using a Freeman Technology FT4 powder rheometer. BFE was analyzed to confirm improved powder flowability of the spray-dried product as compared to the unprocessed water-insoluble inorganic. Lower BFE values are indicative of better confined flow behavior. The results are presented in Table 3.

TABLE 3

| | Unprocessed | Iron Oxide | Unprocessed | Silicon | Unprocessed | Sulfur |
|---|---|---|---|---|---|---|
| | Iron Oxide | Colloid | Silicon | Colloid | Sulfur | Colloid |
| | wt %, solids | wt %, solids | wt %, solids | wt %, solids | wt %, solids | wt %, solids |
| Component | basis | basis | basis | basis | basis | basis |
| Gum Arabic | | 24.24 | | 24.24 | | 24.24 |
| Anti-Foam | | 0.01 | | 0.01 | | 0.01 |
| Iron Oxide | 100 | 75 | | — | | — |
| Silicon | | — | 100 | 75 | | — |
| Sulfur | | — | | — | 100 | 75 |
| Sodium | | 0.75 | | 0.75 | | 0.75 |
| Aluminate | | | | | | |
| Total | | 100.0 | | 100.0 | | 100.0 |
| RESULTS | | | | | | |
| Particle Size | 54.8 | 15.9 | 16.6 | 16.6 | 25.5 | 27.8 |
| D[4, 3], | | | | | | |
| micron | | | | | | |
| Moisture | — | 1.5 | — | 1.6 | — | 2.7 |
| Content, % | | | | | | |
| Basic Flow | 273 | 81 | 73 | 83 | 166 | 118 |
| Energy, mJ | | | | | | |
| CBD, g/mL | 0.80 | 0.75 | 0.75 | 0.64 | 0.66 | 0.63 |

### Example 4

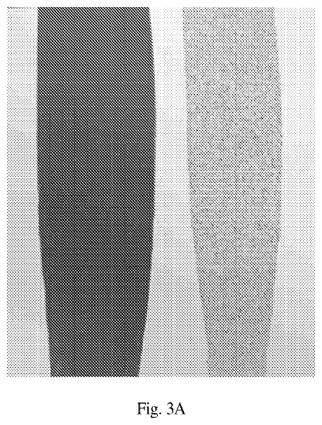

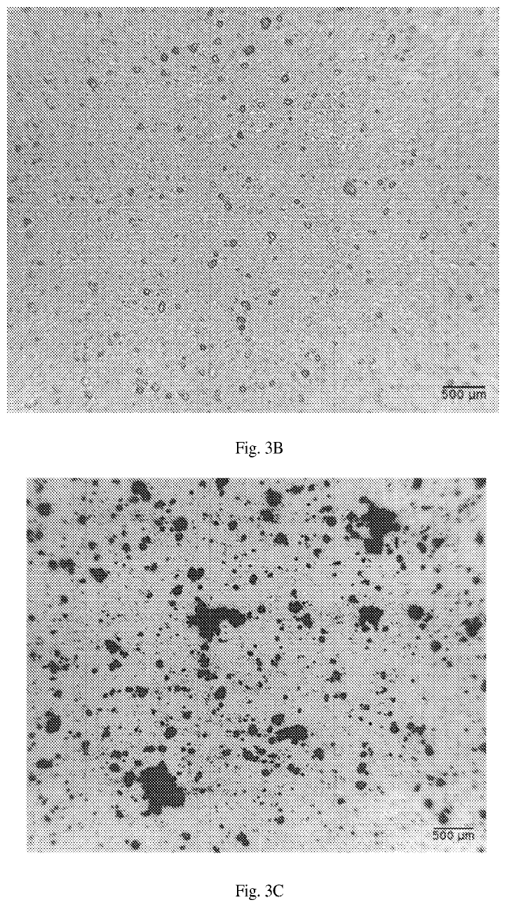

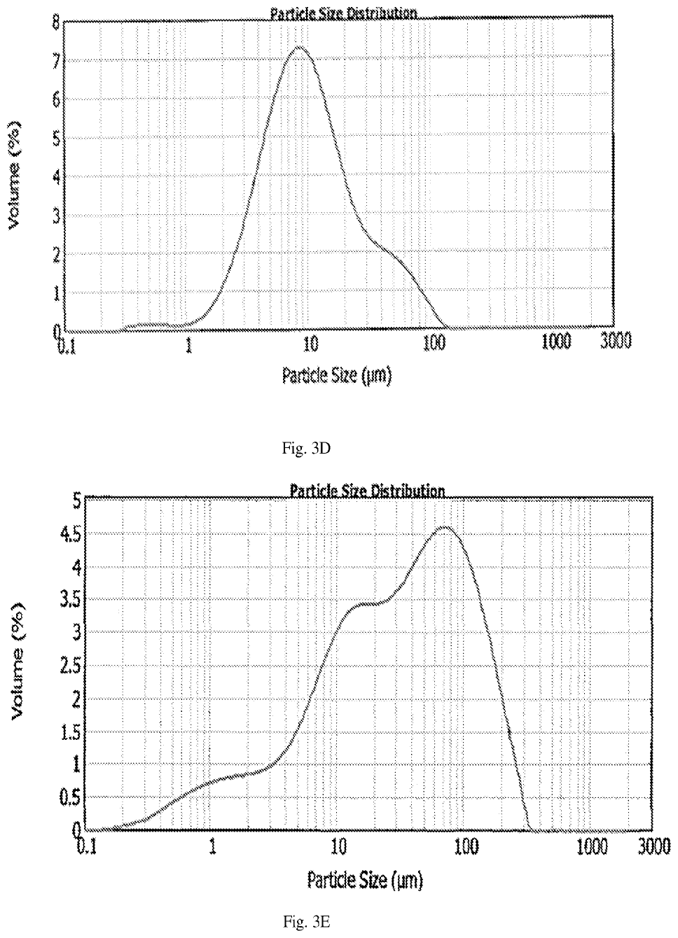

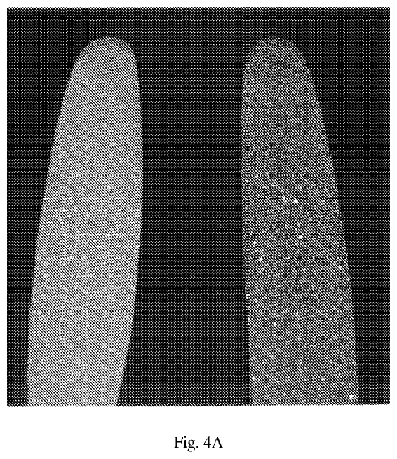

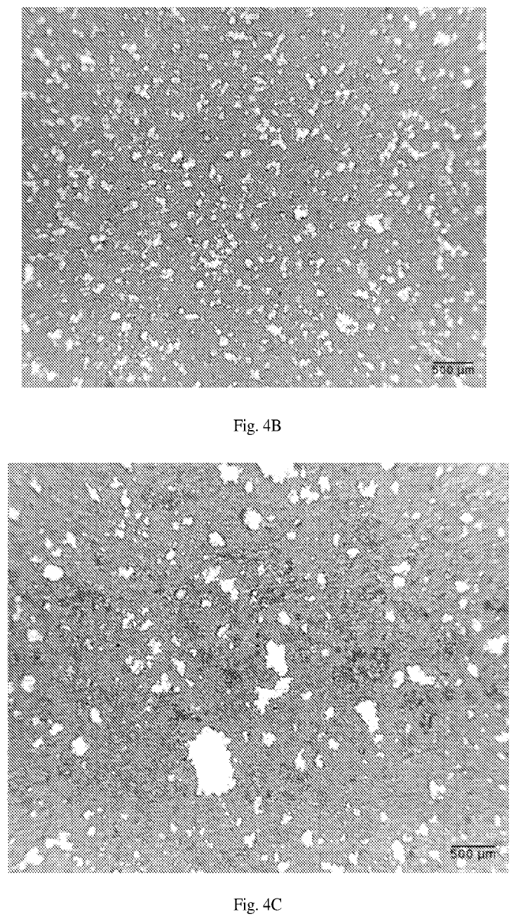

An experiment was conducted to confirm improved dispersability and deagglomeration within a viscous suspension. Suspensions were prepared with both the spray-dried powder products from Example 3 and each unprocessed water-insoluble inorganic under identical processing conditions (Table 4). Suspension G contained a higher amount of spray-dried powder, which only contained 75% of the inorganic, to match the overall inorganic content of the unprocessed water-insoluble inorganic. The resulting suspensions were spread into a thin film using a draw-down bar to compare the dispersability and deagglomeration of each colloid system with its respective control. If the dispersion was clearly not homogeneous and large agglomerates were observed in the draw-down film, the test result was a Fail. See FIGS. 3A-C and 4A-C for images of draw-down films. See FIGS. 3D and E for a comparison of the particle sizes between the iron oxide colloid powder (FIG. 3D) and the iron oxide powder (FIG. 3E).

TABLE 4

| Component | A | B | C | D | E | F | G |
|---|---|---|---|---|---|---|---|
| Water | 81 | 81 | 81 | 81 | 81 | 81 | 79.67 |
| Suspension Base** | 15 | 15 | 15 | 15 | 15 | 15 | 15 |
| Iron oxide | 4 | | | | | | |
| Iron oxide Colloid | | 4 | | | | | |
| Silicon | | | 4 | | | | |
| Silicon Colloid | | | | 4 | | | |
| Sulfur | | | | | 4 | | |
| Sulfur Colloid | | | | | | 4 | 5.33 |
| RESULTS | | | | | | | |
| Draw-down, 10 mil | Fail | Pass | Pass | Pass | Fail | Pass | Pass |
| Approximate | 565 | 143 | 147 | 73.5 | 643 | 132 | 231 |
| diameter of largest | | | | | | | |
| particle in a 28 cm² | | | | | | | |
| area, microns | | | | | | | |

**Suspension base comprised of 13% Glycerin, 3% Xanthan Gum, 21% Cetearyl Alcohol/Ceterath-20 blend, 35% Glyceryl Stearate/PEG-100 Stearate blend, 14% Dicapryl Adipate, 14% Dimethicone.

1. A composition comprising
a. a colloidal material;
b. a water-insoluble inorganic selected from the group consisting of dibasic calcium phosphate, calcium phosphate tribasic, dicalcium sulfate, iron oxide, iron oxide yellow, barium sulfate, silicon, sulfur, and zinc oxide; and
c. a cross-linking agent.

2. The composition of claim 1, wherein the colloidal material is a stabilizer, a demulcent, a binder, an emulsifier, or a texturizer.

3. The composition of claim 1, wherein the colloidal material is selected from the group consisting of gelatin, casein, albumin, agar, dextrin, sugar, welan gum, xanthan gum, guar gum, acacia gum, corn fiber gum, peach gum, locust bean gum, gum rosin, tamarind gum, tragacanth gum, flax seed gum, carrageenan gum, gum arabic, tara gum, and sesbania gum.

4. (canceled)

5. The composition of claim 1, wherein the cross-linking agent is selected from the group consisting of sodium aluminate, calcium aluminate, magnesium aluminate, barium aluminate and strontium aluminate.

6. The composition of claim 1, wherein the composition has a basic flow energy of less than about 150 mJ.

7.-8. (canceled)

9. The composition of claim 1, wherein the composition has a basic flow energy of less than about 85% of a basic flow energy of the water-insoluble inorganic in the absence of the colloidal material and the cross-linking agent.

10. (canceled)

11. The composition of claim 1, wherein the composition has a conditioned bulk density of less than about 97% of a conditioned bulk density of the water-insoluble inorganic in the absence of the colloidal material and the cross-linking agent.

12. (canceled)

13. The composition of claim 1, wherein the composition has a volume moment mean particle size of from about 10 to about 50 microns.

14. (canceled)

15. The composition of claim 1, wherein the water-insoluble inorganic is in an amount of from about 65 wt % to about 85 wt %.

16. (canceled)

17. The composition of claim 1, wherein the colloidal material is present in an amount of from about 13 wt % to about 35 wt %.

18. (canceled)

19. The composition of claim 1, wherein the cross-linking agent is in an amount of from about 0.3 wt % to about 1.5 wt %.

20. (canceled)

21. The composition of claim 1, further comprising water in an amount of from about 1 wt % to about 3 wt %.

22.-25. (canceled)

26. An aqueous composition comprising
a. a colloidal material colloidal material selected from the group consisting of a stabilizer, a demulcent, a binder, an emulsifier, and a texturizer;
b. a water-insoluble inorganic selected from the group consisting of dibasic calcium phosphate, calcium phosphate tribasic, dicalcium sulfate, iron oxide, iron oxide yellow, magnesium oxide, magnesium carbonate, barium sulfate, silicon, sulfur, and zinc oxide;
c. a cross-linking agent selected from the group consisting of sodium aluminate, calcium aluminate, magnesium aluminate, barium aluminate and strontium aluminate; and
d. water.

27. The aqueous composition of claim 26, wherein the water is in an amount of from about 40 wt % to about 70 wt %.

28. (canceled)

29. The aqueous composition of claim 26, wherein the colloidal material is selected from the groups consisting of gelatin, casein, albumin, agar, dextrin, sugar, welan gum, xanthan gum, guar gum, acacia gum, corn fiber gum, peach gum, locust bean gum, gum rosin, tamarind gum, tragacanth gum, flax seed gum, carrageenan gum, gum arabic, tara gum, and sesbania gum.

30.-31. (canceled)

32. The aqueous composition of claim 26, wherein the water-insoluble inorganic is in an amount of from about 19 wt % to about 51 wt %.

33. (canceled)

34. The aqueous composition of claim 26, wherein the colloidal material is present in an amount of from about 4 wt % to about 21 wt %.

35. (canceled)

36. The aqueous composition of claim 26, wherein the cross-linking agent is in an amount of from about 2.0 wt % to about 4.0 wt % relative to the colloidal material.

37. A personal care composition comprising the composition of claim 1, and a base composition.

38.-42. (canceled)

43. A personal care composition comprising the aqueous composition of claim 26, and a base composition.
